(19) United States
(12) Patent Application Publication
(10) Pub. No.: US 2005/0142591 A1
(43) Pub. Date: Jun. 30, 2005

(54) Method of genetic testing in heritable arrhythmia syndrome patients

(76) Inventor: Michael J. Ackerman, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 10/976,086

(22) Filed: Oct. 27, 2004

Related U.S. Application Data
(60) Provisional application No. 60/515,278, filed on Oct. 29, 2003.

(51) Int. Cl. C12Q 1/68; G06F 19/00; G01N 33/48; G01N 33/50

(52) U.S. Cl. 435/6; 702/20

(57) ABSTRACT

A method of diagnosing heritable arrhythmia syndrome in a patient is disclosed. In one embodiment, the method comprises the steps of (a) isolating a nucleic acid sample from the patient and (b) comparing the nucleic acid sample to the compendium of novel DNA mutations disclosed in Table 1, wherein the comparison is to the mutations described from at least one of the genes selected from the group consisting of KCNQ1, KCNH2, SCN5A, and KCNE2.

### CROSS-REFERENCE TO RELATED APPLICATION

This application claims priority to U.S. Ser. No. 60/515,278, filed Oct. 29, 2003 and incorporated by reference herein.

### STATEMENT REGARDING FEDERALLY SPONSORED RESEARCH OR DEVELOPMENT

This invention was made with United States government support awarded by the following agency: National Institutes of Health (HD42569-02). The United States has certain rights in this invention.

### BACKGROUND OF THE INVENTION

Lethal ventricular arrhythmias claim the lives of nearly 400,000 individuals in the United States each year. The majority of these sudden deaths involve middle-aged and elderly persons with coronary artery disease. However, a tragic minority involves previously healthy infants, children, adolescents, and young adults. After excluding structural heart diseases such as hypertrophic cardiomyopathy and anomalous coronary arteries, the most common pathogenic mechanism underlying these unexplained sudden deaths are heritable arrhythmia syndromes or cardiac channelopathies such as congenital long QT syndrome (LQTS).

LQTS affects 1 in 5000 individuals and is characterized by i) a heterogeneous clinical natural history ranging from asymptomatic longevity to sudden death in infancy, ii) a heterogeneous electrocardiographic phenotype ranging from a completely normal resting ECG to extreme QT prolongation with manifest T wave alternans, and iii) heterogeneous genetic underpinnings (Ackerman, M. J., Mayo Clin. Proc. 73:250-269,1998). Since the initial identification of the first LQTS genetic locus on chromosome 11 in 1991 (Keating, M., et al., Science 252:704-706, 1991) to the first identification of LQTS-causing mutations involving the KCNH2-encoded potassium channel (Curran, M. E., et al., Cell 80:795-803, 1995) and the SCN5A-encoded cardiac sodium channel (Wang, Q., et al., Cell 80:805-811, 1995) in 1995, LQTS is understood predominantly as a cardiac channelopathy (Ackerman, M. J., Nature Medicine 10:463-464, 2004; Keating, M. T. and M. C. Sanguinetti, Cell 104:569-580, 2001). To date, over 300 mutations involving 5 genes that encode critical cardiac channel subunits have been reported (Splawski, I., et al., Circulation 102:1178-1185, 2000; Gene Connection for the Heart: a Project of the Study Group on Molecular Basis of Arrhythmias, World Wide Web, 2004). More recently, mutations in ankyrin B have been established as the pathogenic basis for the previously elusive and rare type 4 LQTS (LQT4) (Mohler, P. J., et al., Nature 421:634-639, 2003).

Over the past decade, LQTS genetic testing has been conducted in select few research laboratories as part of IRB-approved genotype-phenotype research studies resulting in numerous novel revelations including: the identification of relatively gene-specific electrocardiographic profiles, gene-specific responses to epinephrine QT stress testing, gene-specific arrhythmogenic triggers and arrhythmogenic temporal states such as swimming, alarm clocks, and the postpartum period, gene-specific responsiveness to beta blocker therapy, gene-directed treatment strategies, and gene-specific risk stratification (Moss, A. J., et al., Circulation 92:2929-2934, 1995; Ackerman, M. J., et al., Mayo Clin. Proc., 2002; Shimizu, W., et al., J. Am. Coll. Cardiol. 41:633-642, 2003; Schwartz, P. J., et al., Circulation 103:89-95, 2001; Moss, A. J., et al., Am. J. Cardiol. 84:876-879, 1999; Ackerman, M. J., et al., Mayo Clin. Proc. 74:1088-1094,1999; Khositseth, A., et al., Heart Rhythm 1:60-64, 2004; Choi, G., et al., Circulation, 2004; Moss, A. J., et al., Circulation 101:616-623, 2000; Priori, S. G., et al., New Engl. J. Med. 348:1866-1874, 2003). However, with genetic testing taking anywhere from 6-24 months to complete and the requirement to disseminate the genetic test information to the study subject rather than to the referring physician, the patients and families afflicted with this potential sudden death syndrome and the physicians evaluating and managing such families have not similarly benefited from the genomic breakthroughs in LQTS.

Recently, however, LQTS genetic testing has become a commercially available, clinical diagnostic test thus representing one of the first comprehensive genetic tests for a cardiac condition akin to BRCA1 and BRCA2 breast cancer genetic testing (Genaissance Pharmaceuticals IPR. Genaissance Pharmaceuticals launches its proprietary FAMILION™ test for genetic mutations associated with sudden cardiac death, PR Newswire, 2004). LQTS genetic testing now joins cystic fibrosis genetic testing as the only ion channel genetic tests currently available clinically. Accordingly, we report the results from our 6-year experience of LQTS genetic testing conducted for 388 consecutive, unrelated patients with a referral diagnosis of LQTS. These observations provide key insights to guide the proper utilization and interpretation of this newly available clinical test.

### BRIEF SUMMARY OF THE INVENTION

In one embodiment, the present invention is a method of diagnosing heritable arrhythmia syndrome in a patient comprising the steps of (a) isolating a nucleic acid sample from the patient and (b) comparing the nucleic acid sample to the compendium of novel DNA mutations disclosed in Table 1, wherein the comparison is to the mutations described from at least one of the genes selected from the group consisting of KCNQ1, KCNH2, SCN5A, and KCNE2.

In preferred versions of the present invention, the comparison is via high throughput DNA sequencing, the nucleic acid sample is from a blood, tissue or buccal smear sample and/or the patient is prior to initiation of medication with known QT prolonging potential.

In other embodiments of the present invention, the comparison is to at least two of the genes, the comparison is to at least three of the genes or the comparison is to all four of the genes.

In another embodiment, the present invention is a method of diagnosing heritable arrhythmia syndrome in a patient comprising the steps of (a) isolating a nucleic acid sample from the patient and (b) comparing the nucleic acid sample to the DNA mutations in gene KCNQ1 listed in Table 1, wherein the comparison is to the mutations listed for at least one of the exons selected from the group consisting of KCNQ1 exons 1, 2, 3, 4, 5, 6, 7, 8, 9, 10, 12, 13, 14, 15 and 16; KCNH2 exons 2, 3, 4, 5, 6, 7, 9, 10, 11, 12, 13, 14 and 15; SCN5A exons 2, 3, 5, 7, 10, 11, 13, 17, 24, 25, 26 and 28; and KCNE2 exon 3.

In another embodiment, the present invention is a method of diagnosing a genetic basis underlying a drug-induced adverse QT event including syncope, aborted cardiac arrest, or sudden death in a patient comprising the steps of (a) isolating a nucleic acid sample from the patient and (b) comparing the nucleic acid sample to the compendium of novel DNA mutations disclosed in Table 1, wherein the comparison is to the mutations described from at least one of the genes selected from the group consisting of KCNQ1, KCNH2, SCN5A, and KCNE2 or a method of performing pre-prescription genotyping in a patient prior to initiation of a medication with known QT prolonging potential comprising the steps of (a) isolating a nucleic acid sample from the patient and (b) comparing the nucleic acid sample to the compendium of novel DNA mutations disclosed in Table 1, wherein the comparison is to the mutations described from at least one of the genes selected from the group consisting of KCNQ1, KCNH2, SCN5A, and KCNE2.

In another embodiment, the present invention is a method of performing pre-prescription genotyping in a patient prior to initiation of a medication with possible or known QT prolonging potential comprising the steps of (a) obtaining a nucleic acid sample from a patient prior to exposure to a medication and (b) comparing a nucleic acid sample to the compendium of novelty in a mutation is disclosed in Table 1, wherein the comparison is to the mutations described from at least one of the genes selected from the group consisting of KCNQ1, KCNH2, SCN5A, and KCNE2, and wherein presence of a novel DNA mutation in the nucleic acid sample indicates that the patient may encounter cardiac risk upon exposure to the medication.

Other objects, features and embodiments of the present invention are apparent to one of skill in the art upon examination of the specification, claims and drawings.

### BRIEF DESCRIPTION OF THE SEVERAL VIEWS OF THE DRAWINGS

FIG. 1. Channel topology of KCNQ1 with LQT1-associated variants. Depicted here is the linear channel topology of the I_{Ks} alpha subunit encoded by KCNQ1 with the approximate location of the pathogenic LQT1-causing mutations indicated. The number within the circle corresponds to the case # on Table 3.

FIG. 2. Channel topology of KCNH2 with LQT2-associated variants. Depicted here is the linear channel topology of the I_{Kr} potassium channel encoded by KCNH2 with the location of the pathogenic LQT2-causing variants indicated. The number within the circle corresponds to the case # on Table 4.

FIG. 3. Channel topology of SCN5A with LQT3-associated variants. Depicted here is the linear channel topology of the NaV1.5 cardiac sodium channel encoded by SCN5A with the location of the pathogenic LQT3-causing variants indicated. The number within the circle corresponds to the case # on Table 5.

FIG. 4. Summary of LQTS genotypes among 388 consecutive, unrelated patients.

FIG. 5. Yield of genetic testing based upon resting QTc and cumulative diagnostic score for LQTS. The likelihood of elucidating a LQTS-associated mutation ranged from 0% when the subject's QTc<420 ms to 62% when the QTc exceeded 480 ms (p<0.001). The greatest yield (75%) was achieved among the subset with a cumulative diagnostic score (“Schwartz score”)≧4 indicating strong probability for the clinical diagnosis of LQTS.

FIG. 6. LQT2-causing KCNH2 mutations and postpartum-triggered cardiac events. Displayed is the linear channel topology of the KCNH2-encoded HERG (I_{Kr}) potassium channel and the location of the pathogenic LQT2-causing mutations. Novel mutations are indicated by *. Missense mutations are indicated with a solid circle, the deletion mutation is indicated by a solid rectangle, and the 4 frameshift mutations culminating in premature truncation are indicated by a filled rectangle followed by a curved line and an octagonal “stop” sign. Note that the R1005fs/50 and the R1033fs/23 mutations terminate at the same residue.

FIG. 7. Gene-specificity of postpartum-triggered cardiac events. Thirteen of 80 unrelated patients (16%) with LQT2 mutations had personal and/or a family history of cardiac events during the postpartum period which was significant greater than 1 of 103 patients (<1%) with LQT1 mutations (P=0.0001).

### DETAILED DESCRIPTION OF THE INVENTION

In one embodiment, the present invention consists of a compendium of novel DNA mutations in novel DNA mutations involving 4 of the 5 cardiac channel genes implicated in congenital long QT syndrome. These DNA mutations are listed below in Table 1. The mutations are listed by the name of the gene, base position and mutational occurrence. For example, the first mutation is a mutation in gene KCNQ1 at base position 153 where a C is replaced by a G. Referring to Table 1, “variant” is a generic description of a genetic alteration that results in an amino acid change which may or may not be a pathogenic mutation. The change could also be a polymorphism.

If one of skill in the art wished to obtain the entire sequence for the gene and the surrounding nucleotide region, one could consult NCBI (National Center for Biotechnology Information), GenBank, or other sequence depositories.

TABLE 1

| | | Base | | | | Mutation/ | |
|---|---|---|---|---|---|---|---|
| Gene | Exon | Position | Nucleotide | Variant | Location | Polymorphism | Novel |
| KCNQ1 | 1 | 153 | 153 C>G | Y51X | N-term | Mutation | YES |
| KCNQ1 | 1 | 217 | 217 C>A | P73T | N-term | Mutation | YES |
| KCNQ1 | 1 | 287 | del287C | S95fs/141 | N-term | Mutation | YES |
| KCNQ1 | 1 | 344 | 344 A>G | E115G | N-term | Mutation | YES |
| KCNQ1 | 1 | 364 | ins T 364-365 | K121fs/629 | N-term | Mutation | YES |
| KCNQ1 | 1 | 365 | 365 G>A | C122Y | N-term | Mutation | YES |
| KCNQ1 | 2 | 397 | 397 G>A | V133I | S1 | Mutation | YES |
| KCNQ1 | 2 | 407 | 407 G>T | C136F | S1 | Mutation | YES |
| KCNQ1 | 3 | 488 | del488T | V162fs/72 | S2 | Mutation | YES |
| KCNQ1 | 4 | 610 | 610 A>T | I204F | S3 | Mutation | YES |
| KCNQ1 | 5 | 704 | 704 T>A | I235N | S4 | Mutation | YES |
| KCNQ1 | 5 | 760 | del 760-768 | Del VVF 254-256 | S4/S5 | Mutation | YES |
| KCNQ1 | 5 | 776 | 776 G>T | R259L | S4/S5 | Mutation | YES |
| KCNQ1 | 6 | 818 | 818 T>G | L273R | S5 | Mutation | YES |
| KCNQ1 | 6 | 826 | del 826-828 | Del S 276 | S5 | Mutation | YES |
| KCNQ1 | 6 | 832 | 832 T>C | Y278H | S5 | Mutation | YES |
| KCNQ1 | 6 | 868 | 868 G>A | E290K | S5-PORE | Mutation | YES |
| KCNQ1 | 6 | 875 | 875 G>A | G292D | S5-PORE | Mutation | YES |
| KCNQ1 | 6 | 877 | 877 C>T | R293C | S5-PORE | Mutation | YES |
| KCNQ1 | 6 | 905 | 905 C>T | A302V | PORE | Mutation | YES |
| KCNQ1 | 6 | 910 | 910 T>C | W304R | PORE | Mutation | YES |
| KCNQ1 | 7 | 940 | 940 G>C | G314R | PORE | Mutation | YES |
| KCNQ1 | 7 | 941 | 941 G>A | G314D | PORE | Mutation | YES |
| KCNQ1 | 7 | 964 | 964 A>C | T322A | PORE/S6 | Mutation | YES |
| KCNQ1 | 7 | 1014 | DEL1014-1016 | DEL F339 | S6 | Mutation | YES |
| KCNQ1 | 7 | 1027 | 1027 C>T | P343S | S6 | Mutation | YES |
| KCNQ1 | 7 | 1031 | 1031 C>A | A344E | S6 | Mutation | YES |
| KCNQ1 | 8 | 1085 | 1085 A>G | K362R | C-term | Mutation | YES |
| KCNQ1 | 9 | 1121 | 1121 T>A | L374H | C-term | Mutation | YES |
| KCNQ1 | 8 | 1124 | del 1124-1127 | L374/fs43 | C-term | Mutation | YES |
| KCNQ1 | 8 | 1128 | 1128 + 1 G>T | Q376sp | C-term | Mutation | YES |
| KCNQ1 | 9 | 1135 | 1135 T>C | W379R | C-term | Mutation | YES |
| KCNQ1 | 9 | 1166 | 1166 C>A | S389Y | C-term | Mutation | YES |
| KCNQ1 | 9 | 1201 | ins C 1201-1202 | P400 fs/62 | C-term | Mutation | YES |
| KCNQ1 | 10 | 1265 | ins A 1265-1266 | K422fs/39 | C-term | Mutation | YES |
| KCNQ1 | 10 | 1343 | insC 1343-1344 | P448fs/13 | C-term | Mutation | YES |
| KCNQ1 | 10 | 1354 | 1354 C>T | R452W | C-term | Mutation | YES |
| KCNQ1 | 12 | 1571 | 1571 T>G | V524G | C-term | Mutation | YES |
| KCNQ1 | 12 | 1576 | 1576 A>G | K526G | C-term | Mutation | YES |
| KCNQ1 | 13 | 1608 | 1608 C>A | Y536X | C-term | Mutation | YES |
| KCNQ1 | 13 | 1637 | 1637 C>T | S546L | C-term | Mutation | YES |
| KCNQ1 | 13 | 1664 | 1664 G>A | R555H | C-term | Mutation | YES |
| KCNQ1 | 14 | 1697 | 1697 C>A | S566Y | C-term | Mutation | YES |
| KCNQ1 | 14 | 1700 | 1700 T>G | I567S | C-term | Mutation | YES |
| KCNQ1 | 15 | 1750 | 1750 G>A | G584S | C-term | Mutation | YES |
| KCNQ1 | 15 | 1768 | 1768 G>A | A590T | C-term | Mutation | YES |
| KCNQ1 | 16 | 1799 | 1799 C>T | T600M | C-term | Mutation | YES |
| KCNQ1 | 16 | 1855 | 1855 T>A | L619M | C-term | Mutation | YES |
| KCNQ1 | 16 | 1876 | 1876 G>A | G626S | C-term | Mutation | YES |
| KCNQ1 | 16 | 2025 | ins G 2025-2026 | G675fs/17 | C-term | Mutation | YES |
| KCNH2 | 2 | 92 | 92 T>G | I31S | N-term | Mutation | YES |
| KCNH2 | 2 | 164 | 164 C>T | S55L | N-term | Mutation | YES |
| KCNH2 | 2 | 254 | 254 C>T | A85V | N-term | Mutation | YES |
| KCNH2 | 2 | 299 | 299 G>A | R100Q | N-term | Mutation | YES |
| KCNH2 | 3 | 395 | Del 395-456 | V131fs/185 | N-term | Mutation | YES |
| KCNH2 | 3 | 453 | ins CC 453-454 | P151 fs/14 | N-term | Mutation | YES |
| KCNH2 | 4 | 545 | 545 C>A | S182X | N-term | Mutation | YES |
| KCNH2 | 4 | 685 | 685G>T | E229X | N-term | Mutation | YES |
| KCNH2 | 4 | 712 | 712 G>A | G238S | N-term | Mutation | YES |
| KCNH2 | 4 | 881 | 881 G>T | G294V | N-term | Mutation | YES |
| KCNH2 | 4 | 916 | 916 G>T | G306W | N-term | Mutation | YES |
| KCNH2 | 5 | 959 | 959 C>T | S320L | N-term | Mutation | YES |
| KCNH2 | 5 | 981 | DEL 981-991 | R326 fs/0 | N-term | Mutation | YES |
| KCNH2 | 5 | 982 | 982 C>T | R328C | N-term | Mutation | YES |
| KCNH2 | 5 | 1121 | ins GC 1121-1122 | V374fs/0 | N-term | Mutation | YES |
| KCNH2 | 6 | 1259 | 1259 A>G | Y420C | S1 | Mutation | YES |
| KCNH2 | 6 | 1262 | 1262 C>T | T421M | S1 | Mutation | YES |
| KCNH2 | 6 | 1264 | 1264 G>A | A422T | S1 | Mutation | YES |
| KCNH2 | 6 | 1280 | 1280 A>C | Y427S | S1-S2 | Mutation | YES |
| KCNH2 | 6 | 1366 | 1366 G>T | D456Y | S2 | Mutation | YES |
| KCNH2 | 6 | 1423 | del TAC 1423-1425 | Y475del | S2/S3 | Mutation | YES |
| KCNH2 | 7 | 1685 | 1685 A>C | H562P | S5 | Mutation | YES |
| KCNH2 | 7 | 1711 | 1711 A>C | I571L | S5 | Mutation | YES |
| KCNH2 | 7 | 1714 | 1714 G>A | G572S | S5 | Mutation | YES |
| KCNH2 | 7 | 1746 | ins GC 1746-1747 | R582fs/11 | S5/PORE | Mutation | YES |
| KCNH2 | 7 | 1787 | 1787 C>G | P596R | S5/PORE | Mutation | YES |
| KCNH2 | 7 | 1868 | 1868 C>T | T623I | PORE | Mutation | YES |
| KCNH2 | 7 | 1883 | 1883 G>T | G628V | PORE | Mutation | YES |
| KCNH2 | 7 | 1904 | 1904 A>T | N635I | PORE/S6 | Mutation | YES |
| KCNH2 | 7 | 1918 | 1918 T>G | F640V | S6 | Mutation | YES |
| KCNH2 | 9 | 2162 | 2162 C>T | P721L | S6-cNBD | Mutation | YES |
| KCNH2 | 9 | 2364 | 2364 G>C | E788D | CNBD | Mutation | YES |
| KCNH2 | 9 | 2398 | 2398 + 5 G>T | L799/sp | CNBD | Mutation | YES |
| KCNH2 | 10 | 2458 | 2458 G>A | G820R | CNBD | Mutation | YES |
| KCNH2 | 10 | 2510 | 2510 A>G | D837G | C-term | Mutation | YES |
| KCNH2 | 10 | 2587 | 2587 C>T | R863X | C-term | Mutation | YES |
| KCNH2 | 11 | 2626 | 2626 G>T | E876X | C-term | Mutation | YES |
| KCNH2 | 11 | 2660 | 2660 G>A | R887H | C-term | Mutation | YES |
| KCNH2 | 12 | 2705 | del C 2705 | Q901fs/71 | C-term | Mutation | YES |
| KCNH2 | 12 | 2728 | del 2728-2762 | P910fs/16 | C-term | Mutation | YES |
| KCNH2 | 12 | 2738 | 2738 C>T | A913V | C-term | Mutation | YES |
| KCNH2 | 12 | 2766 | del 2766G | R922fs/50 | C-term | Mutation | YES |
| KCNH2 | 12 | 2785 | insG 2785-2786 | G928fs/10 | C-term | Mutation | YES |
| KCNH2 | 13 | 2987 | 2987 A>T | N996I | C-term | Mutation | YES |
| KCNH2 | 13 | 3014 | del 3014G | R1005fs/50 | C-term | Mutation | YES |
| KCNH2 | 13 | 3098 | insCG 3098-3099 | R1033fs/23 | C-term | Mutation | YES |
| KCNH2 | 13 | 3101 | del 3101-3108 | R1033fs/81 | C-term | Mutation | YES |
| KCNH2 | 13 | 3103 | DEL 3103 | P1034fs/21 | C-term | Mutation | YES |
| KCNH2 | 13 | 3106 | DUP 3106-3112 | V1038fs/80 | C-term | Mutation | YES |
| KCNH2 | 13 | 3107 | 3107 G>A | G1036D | C-term | Mutation | YES |
| KCNH2 | 14 | 3157 | 3157 G>T | E1053X | C-term | Mutation | YES |
| KCNH2 | 14 | 3168 | INS T 3168 | L1056fs/61 | C-term | Mutation | YES |
| KCNH2 | 14 | 3173 | ins G 3173 | S1057fs/60 | C-term | Mutation | YES |
| KCNH2 | 15 | 3470 | 3470 C>T | P1157L | C-term | Mutation | YES |
| SCN5A | 2 | 52 | 52 C>T | R18W | N-term | Mutation | YES |
| SCN5A | 3 | 373 | 373 G>C | V125L | N-term | Mutation | YES |
| SCN5A | 5 | 553 | 553 G>A | A185T | IS2-IS3 | Mutation | YES |
| SCN5A | 7 | 733 | 733 C>A | Q245K | IS4-IS5 | Mutation | YES |
| SCN5A | 10 | 1211 | 1211 T>A | L404Q | IS6 | Mutation | YES |
| SCN5A | 10 | 1218 | 1218 C>A | N406K | IS6 | Mutation | YES |
| SCN5A | 11 | 1384 | 1384 G>A | E462K | IS6-IIS1 | Mutation | YES |
| SCN5A | 13 | 1910 | 1910 C>T | P637L | IS6-IIS1 | Mutation | YES |
| SCN5A | 13 | 1943 | 1943 C>T | P648L | IS6-IIS1 | Mutation | YES |
| SCN5A | 17 | 2989 | 2989 G>T | A997S | IIS6-IIIS1 | Mutation | YES |
| SCN5A | 17 | 3206 | 3206 C>T | T1069M | IIS6-IIIS1 | Mutation | YES |
| SCN5A | 24 | 3691 | 3691 G>A | E1231K | IIIS1-IIIS2 | Mutation | YES |
| SCN5A | 24 | 4299 | 4299 + 1 G>T | G1433sp | IIIS5-IIIS6 | Mutation | YES |
| SCN5A | 25 | 4373 | 4373 C>A | S1457Y | IIIS6 | Mutation | YES |
| SCN5A | 26 | 4442 | 4442 G>A | G1481E | IIIS6-IVS1 | Mutation | YES |
| SCN5A | 28 | 4999 | 4999 G>A | V1667I | IVS5 | Mutation | YES |
| SCN5A | 28 | 5287 | 5287 G>A | V1763M | IVS6 | Mutation | YES |
| SCN5A | 28 | 5477 | 5477 G>A | R1826H | C-term | Mutation | YES |
| SCN5A | 28 | 5726 | 5726 A>G | Q1909R | C-term | Mutation | YES |
| SCN5A | 28 | 5873 | 5873 G>A | R1958Q | C-term | Mutation | YES |
| KCNE2 | 3 | 40 | 40 G>A | V14I | N-term | Mutation | YES |

We obtained this compendium by mutational analysis using denaturing high performance liquid chromatography (DHPLC) and direct DNA sequencing as performed on genetic material (DNA) obtained from approximately 500 patients referred to Mayo Clinic's Sudden Death Genomics Laboratory because of a suspected channelopathy. These methods are described below in the Examples and in Choi, et al., Circulation 2119-2124, Oct. 12, 2004 and Khositseth, Heart Rhythm 1:60-64, 2004 (both incorporated by reference).

Herein a “novel DNA mutation” is defined as a mutation that results in a structural change in the protein encoded by that change. “Nonsynonymous variants” or “amino-acid-altering variants” are exchangeable terms and are both “novel DNA mutations.” Excluded in this dataset are silent polymorphisms: i.e. DNA mutations that do not alter the sequence of the protein. The novel DNA mutations annotated herein (Table 1) have been demonstrated to be absent among over 1400 reference alleles derived from over 700 healthy subjects from 4 different ethnic groups (Ackerman, M. J., et al., Mayo Clin. Proc. 78:1479-1487, 2003; Ackerman, M. J., et al., Heart Rhythm (in press November 2004)).

In another embodiment, the invention is the use of the mutations in the compendium of Table 1 for diagnostic interpretation of patients, particularly patients believed to be at risk for arrhythmia. In a preferred method, one would obtain a nucleic acid sample from the patient and compare the sequence of the known cardiac channelopathy causing genes with the compendium. For patients suspected of having LQTS, the 5 genes: KCNQ1, KCNH2, SCN5A, KCNE1, and KCNE2, would preferably be analyzed and the findings compared to the compendium of mutations identified herein. For patients suspected of having BrS, the gene, SCN5A, would preferably be analyzed. One would then be able to determine whether any particular novel DNA mutation or mutations exist in the patient and be able to correlate the mutation to risk of arrhythmia.

In a preferred version of the present invention, one would perform the above-identified analysis as follows: A patient, suspected clinically to have a heritable arrhythmia syndrome (“channelopathy”) would submit a blood, tissue, or buccal smear sample for molecular diagnostic testing. Genetic material would be extracted using standard procedures to isolate DNA. Mutational analysis of the suitable genes would typically be performed using high-throughput DNA sequencing. The non-synonymous variant(s) identified would be compared to the compendium of mutations previously implicated as disease-causing mutations of Table 1. The findings from the molecular diagnostic testing will be compared to this compendium through a computer database search and retrieval mechanism. In a preferred form of the present invention, one would compare the patient sample with all the mutations listed in Table 1. In another embodiment of the present invention, one would compare a patient sample with any single one of the mutations listed in Table 1 or with the mutations found in any particular gene listed in Table 1. In another embodiment of the invention, one would compare patient sample with subgroups of Table 1 mutations, preferably mutations in one of the four listed genes or mutations in exons of one the genes. For example, one may wish to compare the nucleic sample of a patient to the mutations listed for exon 1 of KCNQ1.

In another embodiment of the present invention, one would use the compendium described above for pre-prescription genotyping. In a preferred method, one would obtain a nucleic acid sample from a patient who was at possible risk for arrhythmia syndrome or cardiac channelopathies that may be increased or modified by a particular medication. Prior to taking the medication, the patient would be screened for the presence or absence of particular mutations in the compendium. Most preferably, the patient would be specifically screened for mutations known to be adverse to particular pharmaceuticals.

Patients with a history of a drug-induced QT reaction would be subject to a comprehensive molecular diagnostic test as outlined above. In addition, pre-prescription genotyping would be performed on individuals prior to receiving a medication with known QT prolonging potential.

After comparing a test nucleic acid sample to the compendium, an informed diagnostic interpretation could be rendered. Specifically, if a subject referred for molecular diagnostic testing because of a suspected heritable arrhythmia syndrome, a drug-induced adverse QT reaction, or prior to initiation of a drug with QT prolonging potential is found to host a disease-causing variant in Table 1, then a molecular diagnosis of a channelopathy will have been confirmed. Consequently, genotype-directed therapy could be initiated and exposure to such QT prolonging agents would be contraindicated.

### EXAMPLES

### Example 1

### Yield of Genetic Testing in Congenital Long QT Syndrome

Background

Congenital long QT syndrome (LQTS) is a potentially lethal cardiac channelopathy. Over the past decade, LQTS genetic testing has been performed in research laboratories providing numerous genotype-phenotype insights of paramount clinical importance. In May 2004, molecular genetic testing of the 5 common LQTS-causing genes that comprise approximately two-thirds of LQTS became clinically available as a commercial diagnostic test.

Since the discovery that defective cardiac channels provide the pathogenic underpinnings for congenital long QT syndrome (LQTS) in 1995, cardiac channel genetic testing has been performed in research laboratories over the past decade yielding numerous important genotype-phenotype correlations. This study details the prevalence, spectrum, and yield of genetic testing associated with the largest cohort of consecutive, unrelated patients (N=388, 260 females, average age at diagnosis, 23 years, and average QTc, 482 ms) referred to Mayo Clinic's Sudden Death Genomics Laboratory between August 1997 and May 2003 for comprehensive mutational analysis of the 5 cardiac channel genes implicated in LQTS. Overall, 165 putative pathogenic mutations in KCNQ1 (70), KCNH2 (71), SCN5A (23), and KCNE1 (1), were found in 198 unrelated patients (51%). This yield was significantly greater (75%) among the subset with the highest clinical probability of LQTS. The majority of mutations continue to represent novel singletons. The novel mutations identified herein expand the compendium of LQTS-causing mutations by 30%. These observations should help direct physicians to the proper utilization of LQTS genetic testing and should aid in the diagnostic interpretation of the new commercial test.

Methods and Results

A comprehensive cardiac channel gene screen for LQTS-causing mutations in KCNQ1 (LQT1), KCNH2 (LQT2), SCN5A (LQT3), KCNE1 (LQT5), and KCNE2 (LQT6), was performed for 388 consecutive, unrelated patients (260 females, average age at diagnosis, 23 years, and average QTc, 482 ms) referred to Mayo Clinic's Sudden Death Genomics Laboratory for LQTS genetic testing between August 1997 and May 2003. Overall, 165 putative pathogenic mutations in KCNQ1 (70), KCNH2 (71), SCN5A (23), and KCNE1 (1), were found in 198 unrelated patients (51%). Patients with a high clinical probability of LQTS (66/88, 75%) were far more likely to have an identifiable mutation compared to patients with either intermediate (93/215, 43%) or indeterminate (39/85, 46%) clinical probability of LQTS (p<0.0001). Among the 198 genotype positive patients, 177 patients had single pathogenic mutations: LQT1 (88 patients), LQT2 (71), LQT3 (17), and LQT5 (1), and 21 patients (10.6% of genotype-positive patients and 5.3% overall) had 2 possible LQTS-causing mutations. The majority of mutations were novel, missense mutations each identified only once. None of the mutations identified were present in over 1400 reference alleles.

Conclusions

In this comprehensive cardiac channel gene screen of the largest published cohort of consecutive, unrelated patients referred to a research laboratory for LQTS genetic testing, over half of the patients had an identifiable mutation. This yield was significantly greater (75%) among the subset with the highest clinical probability of LQTS. The majority of mutations continue to represent novel singletons. The novel mutations identified herein expand the compendium of LQTS-causing mutations by 30%. These observations should help direct physicians to the proper utilization of LQTS genetic testing and should aid in the diagnostic interpretation of the new commercial test.

Methods

Comprehensive mutational analysis of unrelated LQTS cases. Informed written consent was obtained in accordance with study protocols approved by the Mayo Foundation Institutional Review Board. Between August 1997 and May 2003, 388 consecutive, unrelated patients with a suspected clinical diagnosis of congenital LQTS were referred for LQTS molecular genetic testing at Mayo Clinic's Sudden Death Genomics Laboratory. Regardless of the clinical diagnostic score for LQTS (“Schwartz score”) (Schwartz, P. J., et al., Circulation 88:782-784,1993), a sample was accepted for genetic testing if the referring physician had made a tentative clinical diagnosis of LQTS. Clinical data, including 12-lead ECG, personal history of syncope, seizures, or aborted cardiac arrest, temporally-related triggers, and family history, were extracted and maintained in a custom database, blinded to patient genotype. Sufficient data to derive a clinical “Schwartz” score was available in the majority of cases (n=303, 78%). Differences between continuous variables were assessed using unpaired student t-tests. Nominal variables were analyzed using chi-square analysis. A p-value<0.05 was considered statistically significant.

Patient genomic DNA was analyzed for mutations in all protein-coding exons including splice site regions of the KCNQ1/KVLQT1-encoded I_{Ks} potassium channel alpha subunit (LQT1), the KCNH2/HERG-encoded I_{Kr} potassium channel alpha subunit (LQT2), the SCN5A-encoded cardiac sodium channel channel NaV1.5 (LQT3), the KCNE1/minK-encoded I_{Ks} beta subunit (LQT5), and the KCNE2/MiRP1-encoded I_{Kr} beta subunit (LQT6) using polymerase chain reaction, denaturing high performance liquid chromatography (DHPLC), and automated DNA sequencing (Khositseth, A., et al., supra, 2004; Choi, G., supra, 2004; Splawski, I., et al., Genomics 51:86-97,1998; Ackerman, M. J., et al., Jama 286:2264-2269, 2001).

All putative LQTS-associated variants were denoted using known and accepted nomenclature (Antonarakis, S. E., Human Mutation 11:1-3, 1998). For example, the single letter amino acid code was used to designate non-synonymous, missense variants using the P73T format. Here, at amino acid position 73, the ‘wild type’ amino acid (P=proline) is replaced by a threonine (T) on one of the alleles. To be annotated as a putative LQTS-associated variant, the variant must have involved a conserved residue or splice site that altered the primary amino acid structure of the encoded protein. Hence, synonymous single nucleotide polymorphisms were excluded from consideration. Additionally, to be considered as a putative pathogenic LQTS-causing variant, the non-synonymous variant must have been absent in both published databases of channel polymorphisms and our previous comprehensive analysis of 1488 reference alleles from 4 ethnic groups for the potassium channel genes (Ackerman, M. J., et al., supra, 2003) and 1658 reference alleles for SCN5A (Ackerman, M. J., et al., Heart Rhythm, 2004). As such, the sole or concomitant presence of a common polymorphism such as P448R-KCNQ1, K897T-KCNH2, H558R-SCN5A, or D85N-KCNE1 would not by definition warrant the annotation of LQT1, LQT2, LQT3, or LQT5 respectively and would not be used to assign compound or multiple mutation status to an individual.

Results

Table 2 summarizes the demographics for this cohort of 388 consecutive, unrelated cases (23±16 years, 260 females) having a suspected clinical diagnosis of LQTS. The majority of this cohort was white (89%). There were 15 Hispanic patients (4%), 7 blacks (2%), 3 Asians (0.7%), and 1 Native American. Ethnicity was not available for 16 participants (4%). The average QTc was 482±57 ms and ranged from 368-715 ms. Approximately 25% of the subjects had a QTc exceeding 480 ms and a clinical diagnostic “Schwartz” score≧4 indicating high clinical probability of LQTS. Nearly half of the subjects had fainted and 15% had survived sudden cardiac death. A positive family history was identified in approximately half the cases.

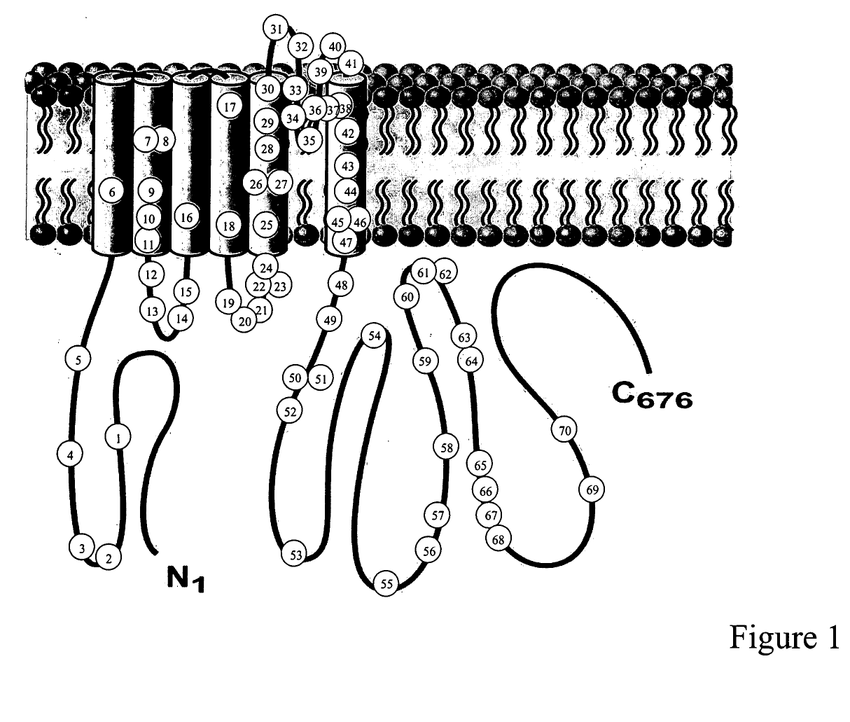

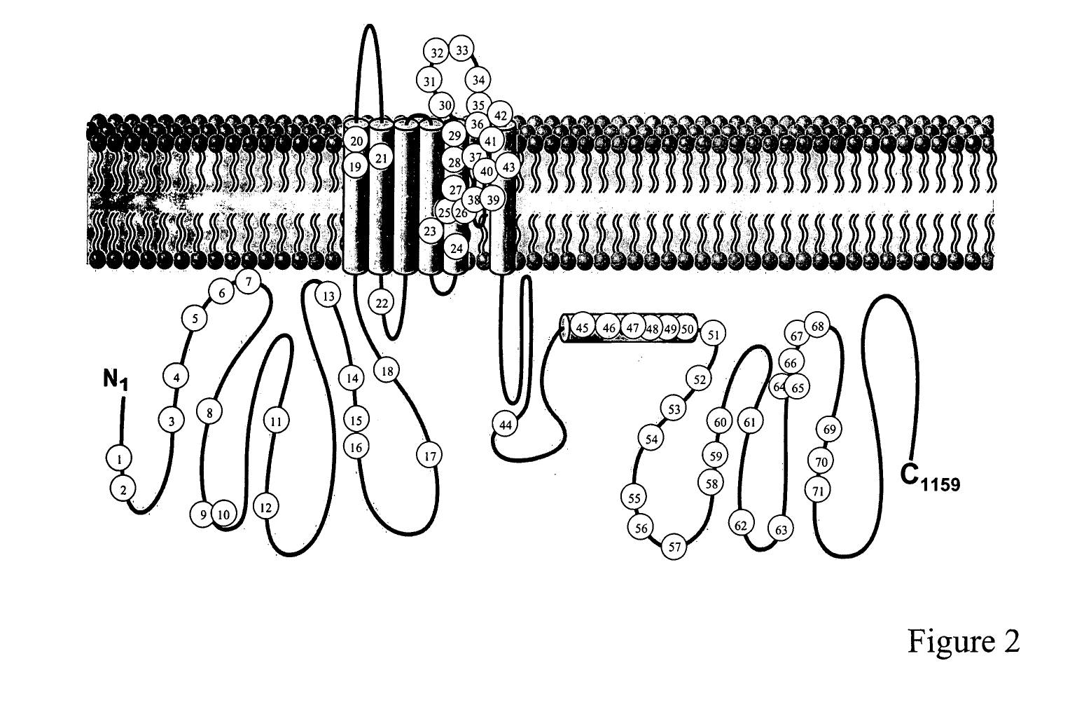

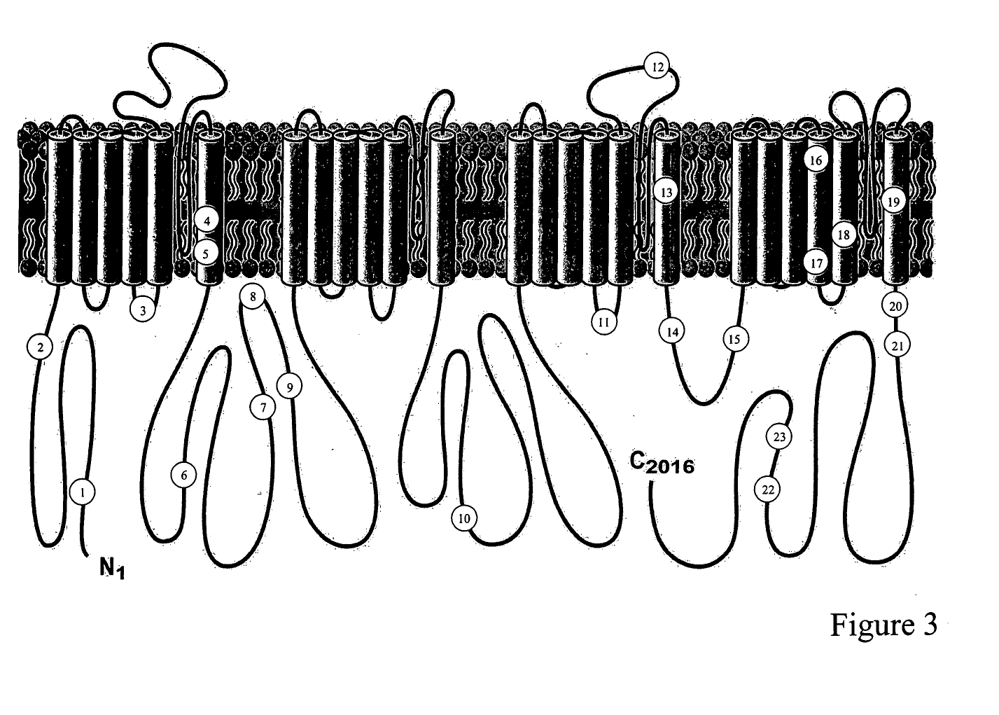

Overall, 165 putative LQTS-causing variants in KCNQ1 (70, Table 3, FIG. 1), KCNH2 (71, Table 4, FIG. 2), SCN5A (23, Table 5, FIG. 3), and KCNE1 (1), were discovered in 198 unrelated patients (51%). Over half of the variants (94/165, 57%) was novel to this cohort including 50%, 63%, and 61% of the KCNQ1, KCNH2, and SCN5A variants respectively. Consistent with the notion of family specific LQTS-causing variants, only 33/165 variants (20%) were observed more than once in this cohort. The 4 most common variants were L191fs/90-KCNQ1 seen in 6 unrelated patients, G269S-KCNQ1 in 4, V524G-KCNQ1 in 6, and T613M-KCNH2 in 4 patients (Tables 2 and 3). The majority of the variants (132/165, 80%) were non-synonymous missense variants. Frame-shift variants comprised 25% of the KCNH2 variants. Only 5 of the 165 variants involved splicing domains. For KCNQ1 variants, most localized to the transmembrane spanning domains (42/70, 60%) rather than the N-terminus (n=5) or C-terminus (n=23, FIG. 1). In contrast, nearly two-thirds of KCNH2 variants localized outside of the transmembrane spanning domains to either the N-terminus (18/71, 25%) or the C-terminus (28/71, 39%, FIG. 2). For the 23 variants in cardiac sodium channel encoded by SCN5A, 2 localized to the N-terminus, 10 to the transmembrane spanning domains, 7 to the inter-domain cytoplasmic linkers, and 4 to the C-terminus (FIG. 3). Within this cohort of unrelated patients, there were no phenotypic distinctions (i.e. degree of QT prolongation or severity of clinical presentation) pursuant to either location or type of mutation (data not shown).

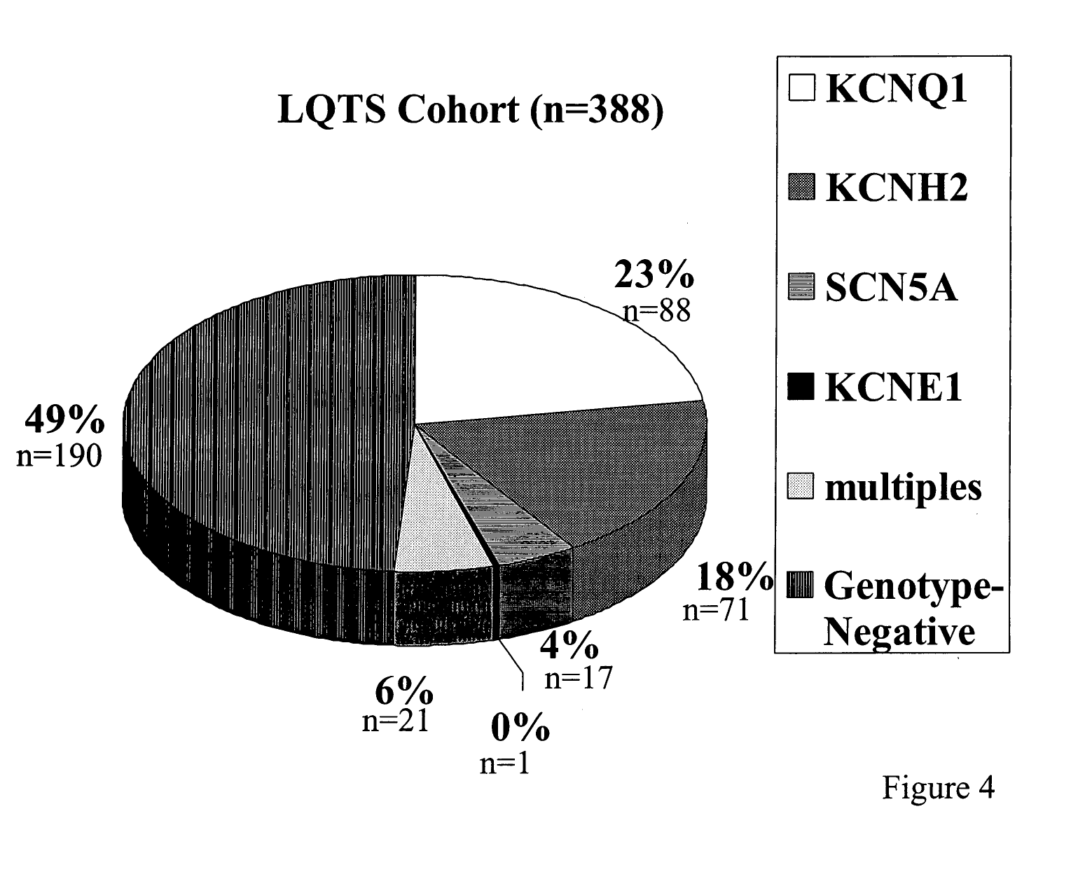

FIG. 4 summarizes the distribution of LQT subtypes for this cohort. Among the 198 genotype positive patients, 177 patients had single pathogenic variants: LQT1 (88 patients), LQT2 (71), LQT3 (17), and LQT5 (1). Twenty-one patients (10.6% of genotype-positive patients and 5.3% overall) had 2 possible LQTS-causing variants: 6 with multiple KCNQ1 variants, 5 with a KCNQ1 and a KCNH2 variant, 4 with a KCNQ1 and a SCN5A variant, 1 with 2 KCNH2 variants, 4 with a KCNH2 and a SCN5A variant, and 1 patient harboring 2 SCN5A variants. Seventeen of the 21 patients harboring multiple variants were white. The QTc (518±62 ms) was greatest among this subset of multiple mutation carriers compared to those hosting a single LQTS-causing variant (494±49 ms, p<0.05) or the 190 patients who were genotype negative 469±54 ms, p<0.002). In addition, the unrelated patients with >1 LQTS-causing variant were younger at diagnosis (16±12 years) than either single mutation individuals (23±17 years) or genotype-negative individuals (24±16 years, p<0.03). There was no difference in the likelihood of a personal history of either syncope or aborted cardiac arrest or a positive family history between those with and those without an identifiable LQTS-causing variant (data not shown).

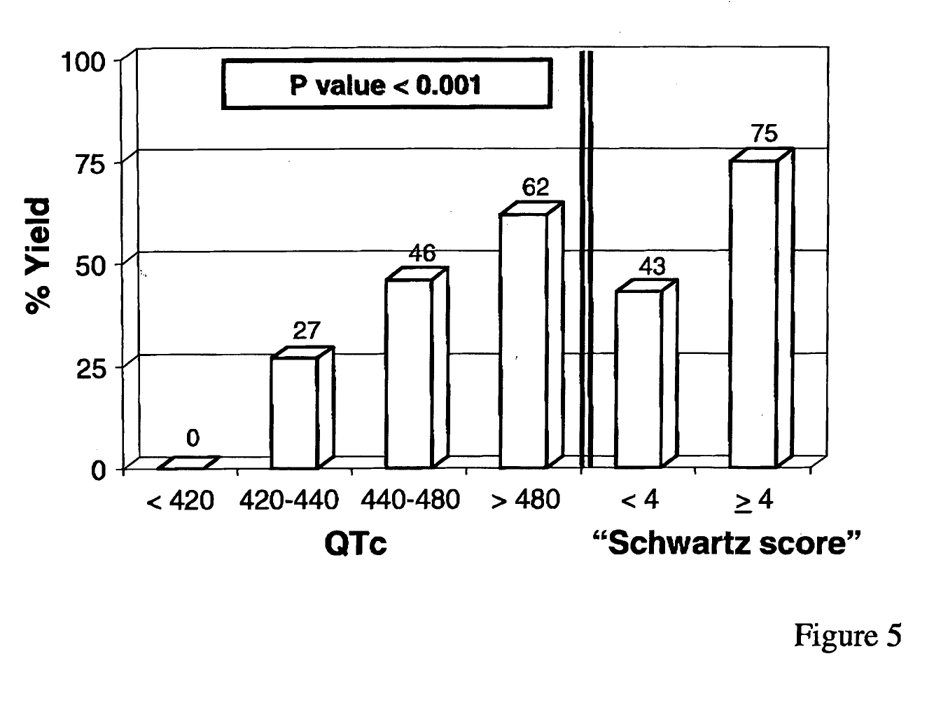

However, the yield from LQTS genetic testing was markedly influenced by the subject's QTc and the cumulative LQTS diagnostic score known as the “Schwartz” score (FIG. 5). 12-lead ECGs were available for independent QTc calculation for 249 subjects. Here, the yield of the genetic test ranged from 0% for the 31 subjects referred with a suspected clinical diagnosis of LQTS despite a resting QTc<420 ms to 62% for the 101 subjects with a screening QTc>480 ms (p<0.0001, FIG. 5). Comprehensive mutational analysis of these 5 cardiac channel genes revealed a LQTS variant for 75% of the subjects who had a “Schwartz” score>4 which indicates a high clinical probability for the syndrome (FIG. 5). Moreover, a definitive genetic diagnosis of LQTS was rendered for 43% of subjects (93/215) who had a composite clinical score that would generally result in the ambiguous clinical diagnosis of so-called “borderline or intermediate probability LQTS”. Although the yield of genetic testing was greater among non-whites than whites (19/26 versus 170/346, p<0.025) with 67% of the 15 Hispanic subjects, 87% of the 7 blacks subjects, and all three Asian subjects hosting an identifiable LQTS-associated variant, ethnicity was not an independent predictor for the genetic test as the clinical phenotype of the non-whites was more suggestive for LQTS (longer QTc, greater “Schwartz” score) than the larger cohort of white cases (data not shown).

Discussion

This study represents the largest series of consecutive, unrelated patients referred for LQTS genetic testing as performed in a research environment. Previously, Splawski and colleagues performed mutational analysis of these 5 LQTS-causing channel genes in 262 unrelated individuals and identified putative LQTS-causing variants in 177 subjects (68%) (Splawski, I., et al., supra, 2000). The difference in overall yield can be accounted for by a careful examination of the 2 cohorts as the probability for a clinical diagnosis of LQTS was higher in the Splawski cohort. The overall QTc was 492±47 ms compared to 482±57 ms in this cohort and LQTS-attributable symptoms were noted in 75% compared to 50% of this cohort. As one might expect, this study confirms that the yield of this cardiac channel gene screen is quite high (75%) when the clinical diagnosis of LQTS is strongly suspected. Conversely, no LQTS-associated mutation has been found in our lab among referred index cases with an accompanying resting QTc<420 ms. Though it is true that relatives of a LQTS case can often be genotype positive yet display a normal QTc reflecting variable expressivity and incomplete penetrance associated with LQTS (so-called concealed LQTS) (Priori, S. G., et al., Circulation 99:529-533, 1999), alternative clinical diagnoses should be considered when this phenotype is ascribed to the index case.

For instance, we demonstrated recently that one mimicker of LQTS, namely catecholaminergic polymorphic ventricular tachycardia (CPVT), is likely dispersed amongst this cohort of patients referred explicitly for LQTS genetic testing (Choi, G., et al., supra, 2004). So far, 9 of the 388 patients (2.3%) in this cohort have a CPVT1-associated mutation involving the RyR2-encoded calcium release channel. These patients all had experienced a personal or family history of a swimming-triggered cardiac event and had a non-diagnostic QTc. We surmise that these patients were suspected of having concealed type 1 LQTS based upon the previous association between LQT1 and swimming (Moss, A. J., et al., supra, 1999; Ackerman, M. J., et al., supra, 1999).

It will be interesting to glean the molecular underpinnings for the remainder of the cohort that remains genotype negative. As CPVT1 analysis has been thus far confined to the subset of 42 patients with a swimming phenotype, the overall prevalence of CPVT1 harbored within this entire cohort of LQTS referrals remains unknown. Already, CPVT1 is far more common in this referred LQTS cohort than LQT5 and LQT6 combined. KCNJ2 mutations responsible for type 1 Andersen-Tawil syndrome (ATS1) (Plaster, N. M., et al., Cell 105:511-519, 2001) have been identified in 5 patients (data not shown). However, all 5 had phenotypic features suggestive of ATS1. Thus, astute recognition of these various cardiac channelopathies such as CPVT and ATS that can masquerade in part as LQTS will be essential to directing the patient's evaluation towards the proper molecular genetic test. Presently, RyR2 and KCNJ2 mutation analysis is not part of the commercial genetic test for cardiac ion channel mutations (Genaissance Pharmaceuticals, supra, 2004).

The prevalence of the first non-cardiac channel genotype of LQTS (LQT4) pursuant to mutations in the Ank2-encoded ankyrin B has not been investigated in this cohort but is likely very rare. Recently, Mohler and colleagues reported that approximately 1% of a cohort of 664 patients selected for Ank2 mutational analysis on the basis of a suspected heritable arrhythmia syndrome had a putative disease-associated mutation and a clinical phenotype of LQTS was seen in only 3 of the Ank2-positive probands (Mohler, P. J., et al., Proc. Natl. Acad. Sci. USA 101:9137-9142, 2004). Finally, novel pathogenic mechanisms surely await discovery as well. In this cohort, 66 of the 88 subjects (75%) having compelling clinical evidence supporting the diagnosis of LQTS had an identifiable mutation. Novel candidate gene exploration and expansion of pedigrees represented by the 22 genotype negative/phenotype positive unrelated cases is currently underway.

A host of genotype-phenotype correlations have been reported in LQTS previously (Moss, A. J., et al., supra, 1995; Ackerman, M. J., et al., supra, 2002; Shimizu, W., et al., supra, 2003; Schwartz, P. J., et al., supra, 2001; Moss, A. J., et al., supra, 1999; Ackerman, M. J., et al., supra, 1999; Khositseth, A., et al., supra, 2004; Choi, G., et al., supra, 2004; Moss, A. J., et al., supra, 2000; Priori, S. G., et al., supra, 2003). Our particular interest in genotype-phenotype has dealt with specific arrhythmogenic triggers. Consistent with previous reports, exertionally-mediated events predominate for LQT1 whereas events in the setting of rest, arousal, or auditory stimuli were more common in LQT2 and LQT3 (data not shown). Previously, we reported from this cohort that there is a strong predilection for swimming-triggered events and LQT1 and events occurring in the postpartum period with LQT2 genotype (Khositseth, A., et al., supra, 2004; Choi, G., et al., supra, 2004). In our cohort, there was no difference in baseline QTc among the 3 most common genotypes. Drawing from only 8 individuals with LQT5 or LQT6, Splawski and colleagues suggested that the QTc might be shorter than the other genotypes (Splawski, I., et al., supra, 2000). Our cohort contained only a single individual with LQT5 and a presenting QTc of 486 ms.

Unrelated individuals harboring multiple mutations presented at a slightly younger age and with a longer QTc than those with a single mutation, findings consistent with a recent report by Westenskow and colleagues on 20 probands with “compound mutation status” (Westenskow, P., et al., Circulation 109:1834-1841, 2004). The prevalence of compound or multiple mutation status was 5.4% for our cohort and 7.9% for the Keating cohort. However, of the 20 probands in the Keating study assigned as having multiple mutations, half possessed the D85N-KCNE1 common polymorphism as the “second hit”. In our study, both variants had to be absent from over 1400 reference alleles to be considered a potential LQTS-associated variant. We have been unable to detect a modifying effect of the common polymorphisms (Ackerman, M. J., et al., supra, 2003; Ackerman, M. J., et al., supra, 2004) such as P448R-KCNQ1, K897T-HERG, H558R-SCN5A, G38S-KCNE1, or D85N-KCNE1 when comparing individuals with a single LQTS-associated variant and those with a single LQTS variant plus a polymorphism in terms of QTc, age at diagnosis, or presence of syncope (data not shown).

At variance with previous studies, we were unable to discern any particular phenotype associated with location of the mutation or type of mutation. Previously, Moss and colleagues demonstrated that within families, patients with pore mutations in HERG did more poorly than those with C-terminal mutations (Moss, A. J., et al., Circulation 105:794-799, 2002). Similar domain-specific phenotypes were reported in LQT1 family studies as well (Shimizu, W., et al., J. Am. Coll. Cardiol. 44:117-125, 2004). In contrast, among unrelated individuals rather than families, we were unable to discern any difference between individuals hosting missense mutations versus frameshift, nonsense, or splicing mutations (data not shown). In addition, the location of the mutation (N-terminus, transmembrane, pore, C-terminus) did not portend any phenotypic distinctions in terms of age at onset, symptoms, or degree of QT prolongation (data not shown).

After nearly a decade of LQTS genetic testing performed in research laboratories, elucidated variants continue to be predominantly novel, family-specific, missense variants. This study adds 94 novel LQTS associated variants to the web-based compendium of catalogued LQTS mutations that currently lists 308 published variants, increasing the compendium by 30% (Gene Connection for the Heart, supra, 2004). Comparing our most recent 100 subjects to the first 100 subjects, the prevalence of novel variants continues to exceed 50% suggesting that saturation of potential LQTS-associated variants has not yet been achieved. The “hottest” mutations in our study were the L191fs/90- and V524G-KCNQ1 variants each found in 6 apparently unrelated individuals. However, formal genetic testing to eliminate the possibility of distant relatedness has not been performed. Excepting KCNH2 which hosts a significant minority (25%) of frameshift and nonsense variants, the vast majority of LQTS variants continue to be non-synonymous single nucleotide substitutions or missense variants. Consequently, comprehensive surveillance of the entire protein-encoding exons of the LQTS-associated genes appears to be the only suitable method of detection for the foreseeable future.

In summary, this comprehensive cardiac channel gene screen of the largest published cohort of consecutive, unrelated patients referred to a research laboratory for LQTS genetic testing revealed an identifiable mutation in over half of the patients. This yield was significantly greater (75%) among the subset with the highest clinical probability of LQTS. The majority of mutations continue to represent novel singletons. The novel mutations identified herein expand the compendium of LQTS-causing mutations by 30%. These observations should help direct physicians to the proper utilization of LQTS genetic testing and should aid in the diagnostic interpretation of the new commercial test. Future studies involving expansion of the pedigrees represented by these unrelated cases will help further define the variability of expression and extent of incomplete penetrance seen for this intriguing channelopathy.

TABLE 2
Demographics of 388 consecutive, unrelated
patients with clinically suspected LQTS

| Age at diagnosis | 23 ± 16 years |
|---|---|
| (range) | (1 day-75 years) |
| Sex (male/female) | 128/260 |
| Ethnicity (% white) | 89 |
| Average QTc (ms) | 482 ± 57 |
| (range) | (368-715) |
| % with QTc > 480 ms | 47 |
| % with syncope | 44 |
| % with cardiac arrest | 14 |
| % with positive family history | 44 |
| % with “Schwartz” score ≧ 4 | 38 |

* QTc = corrected QT interval; ms = milliseconds

TABLE 3
Summary of KCNQ1 LQT1-associated variants

| | | | | | No. of |
|---|---|---|---|---|---|
| Number | Exon | Nucleotide | Variant | Location | Families |
| 1 | 1 | 153 C>G | Y51X* | N-term | 1 |
| 2 | 1 | del 211-219 | AAP71-73del | N-term | 1 |
| 3 | 1 | 217 C>A | P73T* | N-term | 1 |
| 4 | 1 | del C 287 | S95fs/141* | N-term | 1 |
| 5 | 1 | 344 A>G | E115G* | N-term | 1 |
| 6 | 2 | 397 G>A | V133I* | S2 | 1 |
| 7 | 2 | 477 + 5 G>A | M159sp | S2 | 2 |
| 8 | 2 | 477 + 5 G>C | M159sp* | S2 | 1 |
| 9 | 3 | 478 G>A | E160K | S2 | 1 |
| 10 | 3 | del T 488 | V162fs/73* | S2 | 1 |
| 11 | 3 | 502 G>A | G168R | S2 | 2 |
| 12 | 3 | 513 C>G | Y171X | S2-S3 | 1 |
| 13 | 3 | 520 C>T | R174C | S2-S3 | 2 |
| 14 | 3 | 569 G>A | R190Q | S2-S3 | 1 |
| 15 | 3 | del 572-576 | L191fs/90 | S2-S3 | 6 |
| 16 | 4 | 610 A>T | I204F* | S3 | 1 |
| 17 | 4 | 674 C>T | S225L | S4 | 1 |
| 18 | 5 | 704 T>A | I235N* | S4 | 1 |
| 19 | 5 | 724 G>A | D242N | S4-S5 | 1 |
| 20 | 5 | 727 C>T | R243C | S4-S5 | 3 |
| 21 | 5 | 760 G>A | V254M | S4-S5 | 2 |
| 22 | 5 | 775 C>T | R259C | S4-S5 | 1 |
| 23 | 5 | 776 G>T | R259L* | S4-S5 | 2 |
| 24 | 6 | 783 G>C | E261D | S4-S5 | 1 |
| 25 | 6 | 797 T>C | L266P | S5 | 2 |
| 26 | 6 | 805 G>A | G269S | S5 | 4 |
| 27 | 6 | 806 G>A | G269D | S5 | 3 |
| 28 | 6 | del 826-828 | S276del* | S5 | 1 |
| 29 | 6 | 830 C>T | S277L | S5 | 1 |
| 30 | 6 | 832 T>C | Y278H* | S5 | 1 |
| 31 | 6 | 868 G>A | E290K* | S5-PORE | 1 |
| 32 | 6 | 877 C>T | R293C* | S4-S5 | 1 |
| 33 | 6 | 905 C>T | A302V* | PORE | 1 |
| 34 | 6 | 910 T>C | W304R* | PORE | 1 |
| 35 | 7 | 935 C>T | T312I | PORE | 2 |
| 36 | 7 | 940 G>A | G314S | PORE | 2 |
| 37 | 7 | 940 G>C | G314R* | PORE | 1 |
| 38 | 7 | 941 G>A | G314D* | PORE | 2 |
| 39 | 7 | 944 A>G | Y315C | PORE | 1 |
| 40 | 7 | 946 G>A | G316R* | PORE | 1 |
| 41 | 7 | 964 A>G | T322A* | PORE-S6 | 1 |
| 42 | 7 | del 1015-1017 | F339del | S6 | 1 |
| 43 | 7 | 1022 C>T | A341V | S6 | 3 |
| 44 | 7 | 1027 C>T | P343S* | S6 | 1 |
| 45 | 7 | 1031 C>T | A344V | S6 | 3 |
| 46 | 7 | 1032 G>A | A344/G-Asp | S6 | 3 |
| 47 | 8 | 1058 T>C | L353P | S6 | 1 |
| 48 | 8 | 1085 A>G | K362R* | C-term | 1 |
| 49 | 8 | 1096 C>T | R366W | C-term | 1 |
| 50 | 8 | 1121 T>A | L374H* | C-term | 1 |
| 51 | 8 | del 1124-1127 | L374/fs42* | C-term | 1 |
| 52 | 8 | 1128 + 1 G>T | Q376sp* | C-term | 1 |
| 53 | 9 | ins C 1201-1202 | P400fs/61* | C-term | 1 |
| 54 | 10 | 1354 C>T | R452W* | C-term | 1 |
| 55 | 12 | 1552 C>T | R518X | C-term | 3 |
| 56 | 12 | 1571 T>G | V524G* | C-term | 6 |
| 57 | 12 | 1576 A>G | K526E* | C-term | 1 |
| 58 | 12 | 1588 C>T | Q530X | C-term | 1 |
| 59 | 13 | 1615 C>T | R539W | C-term | 3 |
| 60 | 13 | 1637 C>T | S546L* | C-term | 3 |
| 61 | 13 | 1663 C>T | R555C | C-term | 1 |
| 62 | 13 | 1664 G>A | R555H* | C-term | 2 |
| 63 | 14 | 1697 C>A | S566Y* | C-term | 1 |
| 64 | 14 | 1700 T>G | I567S* | C-term | 1 |
| 65 | 15 | 1760 C>T | T587M | C-term | 1 |
| 66 | 15 | 1768 G>A | A590T* | C-term | 1 |
| 67 | 15 | 1772 G>A | R591H | C-term | 1 |
| 68 | 15 | 1781 G>A | R594Q | C-term | 1 |
| 69 | 16 | 1855 T>A | L619M* | C-term | 1 |
| 70 | 16 | 1876 G>A | G626S* | C-term | 1 |

The number in the first column corresponds to the variant's location shown on the channel topology figure (FIG. 1).
*denotes a novel variant, unique to this cohort. Deletion variants are indicated as del, splice site variants are designated by sp, and frameshift mutations are annotated for example as S95fs/141. Here, the last normal amino acid in this 676- amino acid protein is the serine (S) at position 95 followed by 141 “scrambled” amino acids before premature truncation.

TABLE 4
Summary of KCNH2 LQT2-associated variants

| | | | | | No. of |
|---|---|---|---|---|---|
| Number | Exon | Nucleotide | Variant | Location | Families |
| 1 | 2 | 87 C>A | F29L | N-term | 1 |
| 2 | 2 | 92 T>G | I31S* | N-term | 1 |
| 3 | 2 | 164 C>T | S55L* | N-term | 1 |
| 4 | 2 | 193 A>C | T65P | N-term | 1 |
| 5 | 2 | del 221-251 | R73fs/31 | N-term | 1 |
| 6 | 2 | 254 C>T | A85V* | N-term | 1 |
| 7 | 2 | 299 G>A | R100Q* | N-term | 1 |
| 8 | 3 | del 395-456 | V131fs/178* | N-term | 1 |
| 9 | 3 | ins CC 453-454 | P151fs/14* | N-term | 1 |
| 10 | 3 | ins C 453-454 | P151fs/179 | N-term | 2 |
| 11 | 4 | 685 G>T | E229X* | N-term | 1 |
| 12 | 4 | 712 G>A | G238S* | N-term | 1 |
| 13 | 4 | 916 G>T | G306W* | N-term | 1 |
| 14 | 5 | 959 C>T | S320L* | N-term | 1 |
| 15 | 5 | del 981-991 | R326fs/0* | N-term | 1 |
| 16 | 5 | 982 C>T | R328C* | N-term | 2 |
| 17 | 5 | 1096 C>T | R366X* | N-term | 1 |
| 18 | 5 | 1128 G>A | Q376sp | N-term | 1 |
| 19 | 6 | 1262 C>T | T421M* | S1 | 1 |
| 20 | 6 | 1264 G>A | A422T* | S1 | 1 |
| 21 | 6 | 1366 G>T | D456Y* | S2 | 1 |
| 22 | 6 | del TAC 1423-1425 | Y475del* | S2-S3 | 1 |
| 23 | 7 | 1600 C>T | R534C | S4 | 1 |
| 24 | 7 | 1655 T>C | L552S | S5 | 1 |
| 25 | 7 | 1681 G>A | A561T | S5 | 1 |
| 26 | 7 | 1682 C>T | A561V | S5 | 1 |
| 27 | 7 | 1685 A>C | H562P* | S5 | 1 |
| 28 | 7 | 1711 A>C | I571L* | S5 | 1 |
| 29 | 7 | 1714 G>A | G572S* | S5 | 2 |
| 30 | 7 | 1744 C>T | R582C | S5-PORE | 1 |
| 31 | 7 | 1750 G>A | G584S | S5-PORE | 1 |
| 32 | 7 | 1762 A>G | N588D | S5-PORE | 1 |
| 33 | 7 | 1787 C>G | P596R* | S5-PORE | 1 |
| 34 | 7 | 1810 G>A | G604S | S5-PORE | 3 |
| 35 | 7 | 1838 C>T | T613M | PORE | 4 |
| 36 | 7 | 1841 C>T | A614V | PORE | 2 |
| 37 | 7 | 1868 C>T | T623I* | PORE | 1 |
| 38 | 7 | 1882 G>A | G628S | PORE | 2 |
| 39 | 7 | 1883 G>T | G628V* | PORE | 1 |
| 40 | 7 | 1889 T>C | V630A | PORE | 1 |
| 41 | 7 | 1898 A>G | N633S | PORE-S6 | 1 |
| 42 | 7 | 1904 A>T | N635I* | PORE-S6 | 1 |
| 43 | 7 | 1918 T>G | F640V* | S6 | 1 |
| 44 | 9 | 2162 C>T | P721L* | C-term | 1 |
| 45 | 9 | 2320 G>T | D774Y* | C-term | 1 |
| 46 | 9 | 2350 C>T | R784W | C-term | 1 |
| 47 | 9 | 2364 G>C | E788D* | C-term | 1 |
| 48 | 9 | del C 2395 | I798fs/10 | C-term | 1 |
| 49 | 10 | 2414 T>G | F805C* | C-term | 1 |
| 50 | 10 | 2464 G>A | V822M | C-term | 1 |
| 51 | 10 | 2510 A>G | D837G* | C-term | 1 |
| 52 | 10 | 2587 C>T | R863X* | C-term | 1 |
| 53 | 11 | 2626 G>T | E876X* | C-term | 1 |
| 54 | 11 | 2660 G>A | R887H* | C-term | 1 |
| 55 | 12 | del C 2705 | Q901fs/71* | C-term | 1 |
| 56 | 12 | del 2728-2762 | P910fs/16* | C-term | 1 |
| 57 | 12 | 2738 C>T | A913V* | C-term | 2 |
| 58 | 12 | del G 2762 | R920fs/51 | C-term | 1 |
| 59 | 12 | del G 2766 | R922fs/50* | C-term | 1 |
| 60 | 12 | ins G 2785-2786 | G928fs/10* | C-term | 1 |
| 61 | 13 | 2987 A>T | N996I* | C-term | 1 |
| 62 | 13 | del 3014 | R1005fs/50* | C-term | 1 |
| 63 | 13 | 3040 C>T | R1014X | C-term | 1 |
| 64 | 13 | ins CG 3098-3099 | R1033fs/23* | C-term | 1 |
| 65 | 13 | del 3101-3108 | R1033fs/81* | C-term | 1 |
| 66 | 13 | del 3103 | P1034fs/21* | C-term | 1 |
| 67 | 13 | dup 3106-3112 | D1037fs/83* | C-term | 1 |
| 68 | 13 | 3107 G>A | G1036D* | C-term | 1 |
| 69 | 14 | 3157 G>T | E1053X* | C-term | 1 |
| 70 | 14 | ins T 3168 | L1056fs/61* | C-term | 1 |
| 71 | 14 | ins G 3173 | S1057fs/60* | C-term | 1 |

The number in the first column corresponds to the variant's location shown on the channel topology figure (FIG. 2).
*denotes a novel variant, unique to this cohort. Deletion variants are indicated as del, splice site variants are designated by “sp”, and frameshift mutations are designated by “fs”.

TABLE 5
Summary of SCN5A LQT3-associated variants

| | | | | | No. of |
|---|---|---|---|---|---|
| Number | Exon | Nucleotide | Variant | Location | Families |
| 1 | 2 | 52 C>T | R18W* | N-term | 1 |
| 2 | 3 | 373 G>C | V125L* | N-term | 1 |
| 3 | 7 | 733 C>A | Q245K* | IS4-IS5 | 1 |
| 4 | 10 | 1211 T>A | L404Q* | IS6 | 1 |
| 5 | 10 | 1218 C>A | N406K* | IS6 | 1 |
| 6 | 11 | 1384 G>A | E462K* | IS6-IIS1 | 1 |
| 7 | 12 | 1715 C>A | A572D | IS6-IIS1 | 2 |
| 8 | 12 | 1844 G>A | G615E | IS6-IIS1 | 1 |
| 9 | 13 | 1910 C>T | P637L* | IS6-IIS1 | 1 |
| 10 | 17 | 3206 C>T | T1069M* | IIS6-IIIS1 | 1 |
| 11 | 23 | 3974 A>G | N1325S | IIIS4-IIIS5 | 2 |
| 12 | 23 | 4217 G>A | G1406D* | IIIS5-IIIS6 | 1 |
| 13 | 25 | 4370 C>A | S1457Y* | IIIS6 | 1 |
| 14 | 26 | 4442 G>A | G1481E* | IIIS6-IVS1 | 1 |
| 15 | 26 | del 4511-4519 | KPQ 1505-1507del | IIIS6-IVS1 | 1 |
| 16 | 28 | 4868 G>T | R1623L | IVS4 | 1 |
| 17 | 28 | 4931 G>A | R1644H | IVS4 | 2 |
| 18 | 28 | 4999 G>A | V1667I* | IVS5 | 1 |
| 19 | 28 | 5296 A>C | M1766L | IVS6 | 1 |
| 20 | 28 | 5328 G>A | V1777M | C-term | 1 |
| 21 | 28 | 5350 G>A | E1784K | C-term | 2 |
| 22 | 28 | 5726 A>G | Q1909R* | C-term | 1 |
| 23 | 28 | 5873 G>A | R1958Q* | C-term | 1 |

The number in the first column corresponds to the variant's location shown on the channel topology figure (FIG. 3).
*denotes a novel variant, unique to this cohort. Deletion variants are indicated as “del”.

### Example 2

### Identification of a Common Genetic Substrate Underlying Postpartum Cardiac Events in Congenital Long QT Syndrome

The congenital long QT syndrome (LQTS) comprises the first genetically defined type of arrhythmia to be understood at the molecular level as a primary cardiac channelopathy (Ackerman, M. J., et al., N. Engl. J. Med. 336:1575-1586, 1997; Ackerman, M. J., supra, 1998; Keating, M. T. and M. C. Sanguinetti, supra, 2001). To date, 6 LQTS genes have been identified: KCNQ1 (KVLQT1, LQT1), KCNH2 (HERG, LQT2), SCN5A (LQT3), ANKB (Ankyrin-B, LQT4), KCNE1 (mink, LQT5), and KCNE2 (MiRP1, LQT6) (Curran, M., et al., J. Clin. Invest. 92:799-803, 1993; Schoft, J. J., et al., Am. J. Hum. Genet. 57:1114-1122,1995; Sanguinetti, M. C., et al., Nature 384:80-83, 1996; Wang, Q., et al., Nat. Genet. 12:17-23,1996; Bennett, P. B., et al., Nature 376:683-685,1995; Mohler, P. J., et al., supra, 2003). There are relatively gene-specific triggers for cardiac events in LQTS. Patients with LQT1 usually have cardiac events during exercise (62%) whereas LQT2 and LQT3 patients are more likely to have events during rest/sleep (29% and 39%) (Schwartz, P. J., et al., Circulation 103:89-95, 2001). Moreover, swimming appears to trigger events in nearly 15% of children and young adults with symptomatic LQTS and swimming-triggered cardiac events almost universally denote the presence of LQT1 (Garson, A., Jr., et al., Circulation 87:1866-1872, 1993; Moss, A. J., et al., supra, 1999; Ackerman, M. J., et al., supra, 1999). In contrast, the majority of cardiac events triggered by auditory stimuli such as the doorbell and alarm clock occur in patients with LQT2 (Wilde, A. A., et al., J. Am. Coll. Cardiol. 33:327-332, 1999). Rashba and colleagues (Rashba, E. J., et al., Circulation 97:451-456, 1998) reported that the 40 weeks following the birth of a baby are associated with increased risk for cardiac events in women with LQTS but the genetic underpinnings for such postpartum-triggered cardiac events was unknown. The objective of this study was to determine the genetic basis for LQTS in patients with a personal or family history of cardiac events occurring postpartum.

Methods

Study Population

Between August 1997 and May 2003, 388 unrelated patients were referred to Mayo Clinic's Sudden Death Genomics Laboratory for LQTS genetic testing because of a clinical suspicion of LQTS. The study was approved by Mayo Foundation's Institutional Review Board. The presence of a personal and/or family history of cardiac events occurring postpartum was determined by review of the medical records and/or phone interviews and was blinded to the status of genetic testing. In an effort to focus on the time period where postpartum-associated physiological alterations are likely present and to minimize ascertainment/recall bias, the postpartum period was defined as the first 20 weeks following delivery. The standard obstetrical/gynecologic definition of the postpartum period is 4-8 weeks whereas the legal definition for maternal mortality data is the first year following delivery. Cardiac events included sudden cardiac death (SCD), aborted cardiac arrest (ACA), and syncope. Comprehensive mutational analysis of the 5 LQTS-causing channel genes: KCNQ1/KVLQT1 (LQT1), KNCNH21HERG (LQT2), SCN5A (LQT3), KCNE1/mink (LQT5), and KCNE2/MiRP1 (LQT6) was performed using exon-targeted amplification by polymerase chain reaction, denaturing high performance liquid chromatography, and automated DNA sequencing (Ackerman, M. J., et al., Mayo Clin. Proc. 77:413-421, 2002).

Statistical Analysis

All continuous variables were reported as the mean±standard deviation (SD). A two-tailed Fisher's exact test was used to compare the prevalence of the cardiac events during postpartum in each gene mutation. A P value <0.05 was considered to be statistically significant.

Results

Among this cohort of 388 unrelated patients (260 females, average age at diagnosis, 23 years, and average QTc, 481 ms), referred for mutational analysis of the LQTS-causing channel genes because of a clinical diagnosis of suspected LQTS, 14 patients (3.6%) had a personal and/or family history of least one cardiac event during the defined postpartum period (Table 6). Four of these 14 index cases experienced postpartum cardiac events including: appropriate implantable cardioverter-defibrillator (ICD) therapy to terminate ventricular fibrillation during sleep at 4 and 20 weeks postpartum (case 12, Table 6), ACA resulting in profound neurological injury at 16 weeks postpartum (case 1, Table 6), and syncope at 8 weeks postpartum in 2 patients (cases 9 and 10). Eleven of 14 probands had a positive family history of a postpartum-triggered cardiac event: SCD in 7 including 5 first-degree relatives (either mother or sister), ACA in 2, and syncope in 2. The average time from delivery to a cardiac event was 10.5±5.2 weeks (range 1 hour to 20 weeks, median 8 weeks, and mode 8 weeks).

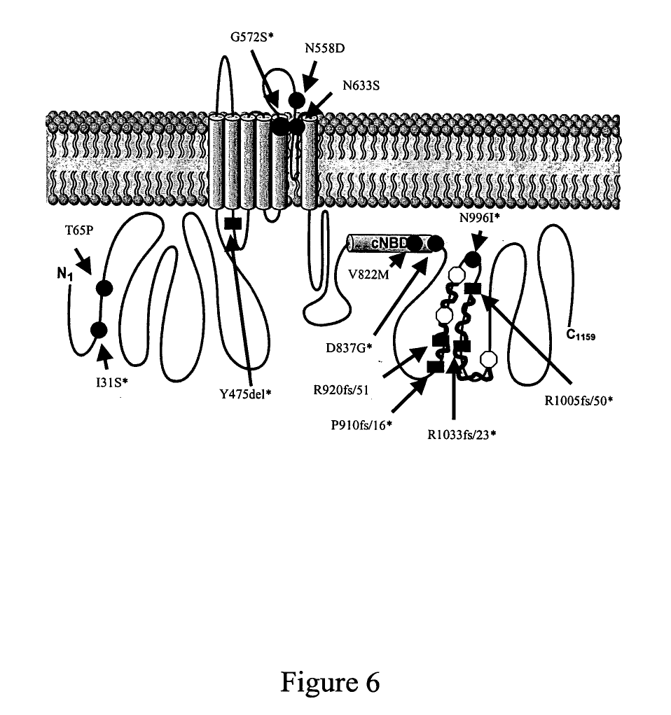

Thirteen of the 14 postpartum-positive probands (93%) harbored mutations in KCNH2 (LQT2) including 8 novel mutations and 5 previously published mutations. One individual (7%) had a novel pore mutation in KCNQ1 (LQT1). Four of the 13 KCNH2 mutations localized to either the channel pore or transmembrane spanning domains while 9 resided in the cytoplasmic N- or C-terminal regions (non-pore regions, Table 6, FIG. 6). The severity of cardiac events (aborted cardiac arrest or sudden cardiac death vs syncope) was not significantly different between non-pore and pore mutations in the KCNH2-encoded HERG potassium channel (data not shown). None of the mutations identified were observed in over 1400 reference alleles (Ackerman, M. J., et al., supra, 2003).

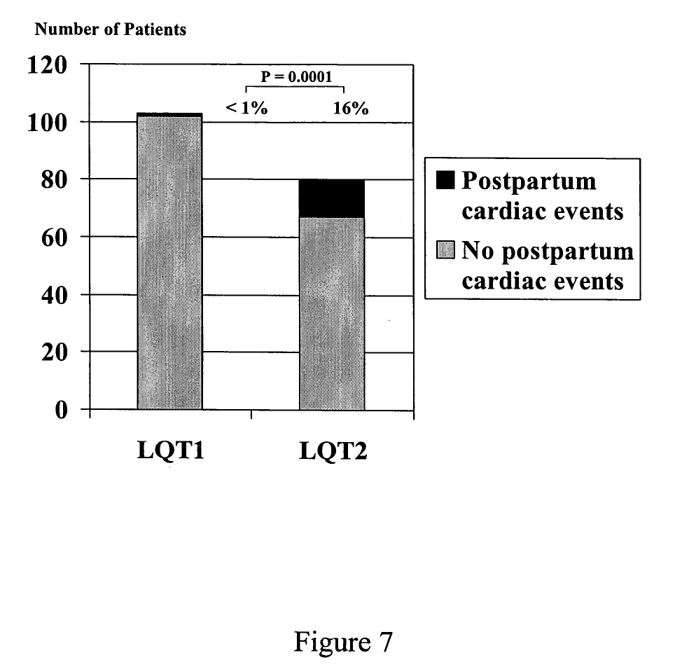

Overall, 13 of the 80 index cases (16%) genotyped for LQT2 had a positive history of a cardiac event postpartum compared to 1 of 103 index cases with LQT1 and none of the remaining genotype positive individuals. Thus, the gene specificity of cardiac events during postpartum period in probands or family members was significantly greater in patients with LQT2 genotype than LQT1 genotype (16% vs <1%, P=0.0001) in this study cohort (FIG. 7).

Discussion

Although bringing in a new life is typically associated with great anticipation and excitement, new mothers with LQTS also enter into a period of increased vulnerability for a life-threatening arrhythmia during this postpartum period (Rashba, E. J., et al., Circulation 97:451-456,1998). Among this cohort of 388 unrelated patients referred for LQTS genetic testing, nearly 4% had a positive personal and/or family history of a postpartum cardiac event. Of the 260 females referred for LQTS genetic testing, 4 (1.5%) have had and survived a postpartum cardiac event. Over 90% of this postpartum-positive subset was found to harbor mutations in KCNH2 responsible for LQT2.

KCNH2 (HERG; chromosome 7q35-36) encodes the alpha subunit underlying delayed rectifier potassium channels (I_{Kr}) in the heart that mediate phase 3 repolarization (Trudeau, M. C., et al., Science 269:92-95,1995; Sanguinetti, M. C., et al., Cell 81:299-307,1995). Mutations of the HERG channel result in decreased I_{Kr} as the electrophysiologic phenotype in LQT2 patients (January, C. T., et al., J. Cardiovasc. Electrophysiol. 11:1413-1418, 2000). Previously, Moss and colleagues reported that patients with mutations in the channel pore of HERG had a more severe phenotype than those harboring non-pore mutations (Moss, M. T., et al., supra, 2002). In our study, the majority of postpartum-positive LQT2 patients had non-pore mutations despite their severe phenotype underscoring the profound heterogeneity in the clinical expression of LQTS.

Precisely why the postpartum period is preferentially arrhythmogenic to those with an underlying LQT2 substrate is unknown. Previously, Rashba and colleagues (Rashba, E. J., et al., supra, 1998) reported pregnant women with LQTS and found that the 40 weeks following delivery of a baby posed a far greater risk for cardiac events than either the 40 weeks of pregnancy or the 40 weeks prior to conception. However, the pathogenetic mechanism underlying this association was unknown.

The psychological stress, changes in sex hormone levels, lactation, alteration of sleep pattern, alteration of life style related to taking care of baby, and abrupt, intense new auditory stimuli (i.e. a crying baby) that are present postpartum may provide arrhythmogenic trigger(s) to women with LQT2. Generally, females have faster resting heart rates and longer QTc than males (Rautaharju, P. M., et al., Can. J. Cardiol. 8:690-695,1992) and a higher risk for syncope and sudden death in LQTS (Moss, A. J., et al., Circulation 84:1136-1144,1991). Estrogen and progesterone may be arrhythmogenic (Romhilt, D. W., et al., Am. J. Cardiol. 54:582-586, 1984) and may play a critical role in cardiac repolarization (Drici, M. D., et al., Circulation 94:1471-1474, 1996). Changes involving the sex hormones estrogen, progesterone, and prolactin during pregnancy and postpartum period could potentially increase the risk of cardiac events. However, levels of estrogen and progesterone are extremely low postpartum and would not likely mediate this LQT2 predilection for postpartum cardiac events. With respect to prolactin, Altemus and colleagues (Altemus, M., et al., Circulation 106:1488-1492, 2002) demonstrated that lactating women had increased vagal contribution to heart rate regulation, and postpartum women who were not lactating had evidence of elevated sympathetic and decreased parasympathetic nervous system activity.

Lanfranchi and colleagues (Lanfranchi, P. A., et al., Circulation 106:1488-1492, 2002) demonstrated a divergent sex-related effect on the RR interval during rapid eye movement (REM) sleep with women having an accentuated QTc during REM compared to men. Sleep disturbance was greatest during the first postpartum month, particularly for first-time mothers and there was improvement in sleep characteristics by the third month postpartum (Lee, K. A., et al., Obstet. Gynecol. 95:14-18, 2000). These two findings, perhaps, explain why most cardiac events occurred around 8 weeks postpartum in our study. Finally, auditory stimuli such as alarm clock triggers cardiac events preferentially in patients with LQT2 (Wilde, A. A., et al., supra, 1999; Wellens, H. J., et al., Circulation 46:661-665, 1972; Nakajima, T., et al., Jpn. Circ. J. 59:241-246, 1995). Akin to an alarm clock, we speculate that perhaps a babies cry startling a LQT2 women during REM sleep may be arrhythmogenic. This speculation is buttressed by observations by Shimizu and Anzelevitch (Shimizu, W. and C. Antzelevitch, J. Am. Coll. Cardiol. 35:778-786, 2000) whereby beta-adrenergic stimulation transiently increased action potential duration, transmural dispersion, and the incidence of torsades de pointes in a pharmacological in vitro model of LQT2.

Study Limitations

Firstly, although we extensively reviewed all sources of data including phone interviews and medical records, it is possible that the ˜4% prevalence of cardiac events occurring postpartum is an underestimate. Importantly, the genetic testing was performed independent of a subject's postpartum phenotype minimizing the potential for bias. Secondly, because of the unavailability of appropriately archived tissue, a molecular autopsy (Ackerman, M. J., et al., N. Engl. J. Med. 341:1121-1125, 1999) was not performed on each decedent who experienced sudden cardiac death postpartum to confirm the presence of the same pathogenic LQTS-causing mutation established in the living probrand in our cohort. However, the LQTS-causing mutation has been confirmed in 6 of the 10 positive family history only cases (cases 2, 5, 6, 8, 13, and 14) by molecular autopsy or determination of its obligate presence through the subsequent voluntary participation of relatives to the index case. Although it seems quite reasonable to assume that the decedent shared the same mutation in the remaining cases, we can not exclude the possibility of a non-LQTS sudden death such as pulmonary thromboembolism or the possibility that other LQTS-causing mutations or channel polymorphisms may have been additionally present in the decedent.

Conclusion

Approximately four percent of this LQTS cohort had a positive history of a cardiac event during the postpartum period, most commonly during the first 2 months after delivery. Mutations in KCNH2 (LQT2) were present in the majority of families experiencing postpartum sudden death, aborted cardiac arrest or syncope. Along with swimming and LQT1 and auditory triggers and LQT2, this association between postpartum cardiac events and LQT2 can facilitate strategic genotyping. The precise triggers that render a woman with LQT2 susceptible to a life-threatening arrhythmia after giving birth warrant further investigation.

TABLE 6
Fourteen index cases who experienced personal and/or family
history of cardiac events during postpartum period

| | Relation- | No. of | Type of | Time from | | | |
|---|---|---|---|---|---|---|---|
| Case | ship to | Cardiac | Cardiac | Delivery | LQTS | | |
| No. | Case | Events | Event | (weeks) | genotype | Mutation | Location |
| 1 | Self | 1 | ACA | 16 | LQT2 | I31S* | N-terminal |
| 2 | FM(1) | 1 | S | 8 | LQT2 | T65P | N-terminal |
| 3 | FM(1) | 1 | SCD | 8 | LQT2 | Y475del* | S2/S3 |
| 4 | FM(1) | 1 | SCD | 8 | LQT2 | G572S* | S5 |
| 5 | FM(1) | 1 | ACA | 16 | LQT2 | N588D | S5/Pore |
| 6 | FM(1) | 1 | SCD | 12 | LQT2 | N633S | Pore/S6 |
| 7 | FM(2) | 1 | SCD | 12 | LQT2 | V822M | CNBD |
| 8 | FM(1) | 1 | S | 6 | LQT2 | D837G* | C-terminal |
| 9 | self^{†} | 2 | S/SCD | 8, 0^{‡} | LQT2 | P910fs/16* | C-terminal |
| 10 | Self | 2 | S/S | 8, 12 | LQT2 | R920fs/51 | C-terminal |
| 11 | FM(2) | 1 | ACA | 8 | LQT2 | N996I* | C-terminal |
| 12 | Self | 2 | S/ACA# | 4, 20 | LQT2 | R1005fs/50* | C-terminal |
| 13 | FM(1) | 1 | SCD | 10 | LQT2 | R1033fs/23* | C-terminal |
| 14 | FM(1) | 1 | SCD | 20 | LQT1 | T322A* | Pore/S6 |

ACA = Aborted cardiac arrest
FM = Family member, number in ( ) indicates relatedness to the index case. (1) denotes a first-degree relative, either mother or sister. (2) indicates a second-degree relative, either aunt or niece
LQTS = Long QT syndrome
S = Syncope
SCD = Sudden cardiac death
*Denotes a novel LQTS-causing mutation
#This patient received an appropriate shock by an implantable cardioverter defibrillation due to ventricular fibrillation
^{†}This patient also had a second-degree relative (maternal aunt) with postpartum sudden cardiac death
^{‡}Cardiac events occurred 1 hour after delivery

1. A method of diagnosing heritable arrhythmia syndrome in a patient comprising the steps of
(a) isolating a nucleic acid sample from the patient and
(b) comparing the nucleic acid sample to the compendium of novel DNA mutations disclosed in Table 1,
wherein the comparison is to the mutations described from at least one of the genes selected from the group consisting of KCNQ1, KCNH2, SCN5A, and KCNE2.

2. The method of claim 1, wherein the comparison is via high throughput DNA sequencing.

3. The method of claim 1, wherein the nucleic acid sample is from a blood, tissue or buccal smear sample.

4. The method of claim 1, wherein the patient is prior to initiation of medication with known QT prolonging potential.

5. The method of claim 1, wherein the comparison is to at least two of the genes.

6. The method of claim 1, wherein the comparison is to at least three of the genes.

7. The method of claim 1, wherein the comparison is to all four of the genes.

8. A method of diagnosing heritable arrhythmia syndrome in a patient comprising the steps of
(a) isolating a nucleic acid sample from the patient and
(b) comparing the nucleic acid sample to the DNA mutations in gene KCNQ1 listed in Table 1, wherein the comparison is to the mutations listed for at least one of the exons selected from the group consisting of KCNQ1 exons 1, 2, 3, 4, 5, 6, 7, 8, 9, 10, 12, 13, 14, 15 and 16.

9. A method of diagnosing heritable arrhythmia syndrome in a patient comprising the steps of
(a) isolating a nucleic acid sample from the patient and
(b) comparing the nucleic acid sample to the DNA mutations in gene KCNH2 listed in Table 1, wherein the comparison is to the mutations listed for at least one of the exons selected from the group consisting of KCNH2 exons 2, 3, 4, 5, 6, 7, 9, 10, 11, 12, 13, 14, and 15.

10. A method of diagnosing heritable arrhythmia syndrome in a patient comprising the steps of
(a) isolating a nucleic acid sample from the patient and
(b) comparing the nucleic acid sample to the DNA mutations in gene SCN5A listed in Table 1, wherein the comparison is to the mutations listed for at least one of the exons selected from the group consisting of SCN5A exons 2, 3, 5, 7, 10, 11, 13, 17, 24, 25, 26 and 28.

11. A method of diagnosing heritable arrhythmia syndrome in a patient comprising the steps of
(a) isolating a nucleic acid sample from the patient and
(b) comparing the nucleic acid sample to the DNA mutations in gene KCNE2 listed in Table 1.

12. A method of diagnosing a genetic basis underlying a drug-induced adverse QT event including syncope, aborted cardiac arrest, or sudden death in a patient comprising the steps of
(a) isolating a nucleic acid sample from the patient and
(b) comparing the nucleic acid sample to the compendium of novel DNA mutations disclosed in Table 1,
wherein the comparison is to the mutations described from at least one of the genes selected from the group consisting of KCNQ1, KCNH2, SCN5A, and KCNE2.

13. A method of performing pre-prescription genotyping in a patient prior to initiation of a medication with known QT prolonging potential comprising the steps of
(a) isolating a nucleic acid sample from the patient and
(b) comparing the nucleic acid sample to the compendium of novel DNA mutations disclosed in Table 1,
wherein the comparison is to the mutations described from at least one of the genes selected from the group consisting of KCNQ1, KCNH2, SCN5A, and KCNE2.

14. A method of performing pre-prescription genotyping in a patient prior to initiation of a medication with possible or known QT prolonging potential comprising the steps of
(a) obtaining a nucleic acid sample from a patient prior to exposure to a medication and
(b) comparing a nucleic acid sample to the compendium of novelty in a mutation is disclosed in Table 1, wherein the comparison is to the mutations described from at least one of the genes selected from the group consisting of KCNQ1, KCNH2, SCN5A, and KCNE2, and wherein presence of a novel DNA mutation in the nucleic acid sample indicates that the patient may encounter cardiac risk upon exposure to the medication.
